(19) United States
(12) Patent Application Publication
(10) Pub. No.: US 2021/0047205 A1
(43) Pub. Date: Feb. 18, 2021

(54) ULTRAVIOLET STERILIZATION TUBE AND ULTRAVIOLET STERILIZATION DEVICE

(71) Applicant: Enplas Corporation, Saitama (JP)

(72) Inventor: Masato NAKAMURA, Saitama (JP)

(73) Assignee: Enplas Corporation, Saitama (JP)

(21) Appl. No.: 16/966,897

(22) PCT Filed: Jan. 31, 2019
(86) PCT No.: PCT/JP2019/003271

§ 371 Date: Aug. 2, 2020

(30) Foreign Application Priority Data
Feb. 2, 2018 (JP) 2018-017568

(51) Int. Cl. C02F 1/32

(52) U.S. Cl. CPC C02F 1/32; C02F 2303/04; C02F 2201/3222

(57) ABSTRACT

This ultraviolet sterilization tube comprises: a channel tube which internally has a processing channel; and a window which is arranged in at least a part of the tube wall of the channel tube. The window comprises: a fluorescent layer which contains a phosphor that emits visible light when irradiated with ultraviolet light; and an ultraviolet light blocking layer which is arranged more distant from the processing channel than the fluorescent layer, and which blocks ultraviolet light, while transmitting visible light.

### TECHNICAL FIELD

The present invention relates to an ultraviolet sterilizing pipe and an ultraviolet sterilizing apparatus.

### BACKGROUND ART

It is widely known to use ultraviolet radiation to sterilize fluids such as liquids. For example, Patent Literature (hereinafter, referred to as “PTL”) 1 describes a fluid sterilization apparatus that directs ultraviolet radiation in the axial direction of a channel onto the channel extending in the axial direction, so as to sterilize a liquid flowing through the channel.

Specifically, the fluid sterilization apparatus described in PTL 1 includes a channel pipe that demarcates a treatment channel extending in the axial direction, and a wide-orientation-angle light emitting element (LED light source) that is disposed in the vicinity of one end portion of the channel pipe and emits ultraviolet radiation in the axial direction from the one end toward the treatment channel. The ultraviolet radiation emitted from the light source sterilizes a fluid flowing through the treatment channel.

### CITATION LIST

### Patent Literature

### PTL 1

### Japanese Patent Application Laid-Open No. 2017-104230

### SUMMARY OF INVENTION

### Technical Problem

Since the wavelength of the ultraviolet radiation used for sterilizing a fluid is 200 nm to 300 nm, the presence or absence of the ultraviolet radiation emitted from the light source cannot be visually confirmed. In this case, it may be considered to confirm the presence or absence of the ultraviolet radiation with a sensor such as an illuminometer. However, if a sensor is used, the apparatus may become complicated and large, and the manufacturing cost may increase. Further, ultraviolet radiation has a shorter wavelength and higher energy than visible light. For this reason, when the human body is exposed to ultraviolet radiation, a danger such as blindness may occur.

Accordingly, it is an object of the present invention to provide an ultraviolet sterilizing pipe and an ultraviolet sterilizing apparatus with which whether or not ultraviolet radiation is radiated to a treatment channel can be confirmed simply and safely.

### Solution to Problem

An ultraviolet sterilization pipe in relation to the present invention for solving the above-described technical problem includes: an ultraviolet sterilization pipe used for irradiating a fluid flowing through a treatment channel with ultraviolet radiation to sterilize the fluid; a channel pipe having the treatment channel therein; a window arranged on at least a part of a pipe wall of the channel pipe; a fluorescent layer including a phosphor that emits visible light when irradiated with the ultraviolet radiation; and an ultraviolet blocking layer that is arranged at a position farther from the treatment channel than the fluorescent layer is and that blocks the ultraviolet radiation and transmits the visible light.

In addition, an ultraviolet sterilization apparatus in relation to the present invention for solving the above-described technical problem includes the ultraviolet sterilization pipe described above and a light source that emits ultraviolet radiation toward the treatment channel.

### Advantageous Effects of Invention

According to the present invention, it is possible to simply and safely confirm whether or not ultraviolet radiation are radiated into the treatment channel.

### BRIEF DESCRIPTION OF DRAWINGS

FIG. 1 is a sectional view of an ultraviolet sterilization apparatus according to Embodiment 1 of the present invention;

FIG. 2 is a sectional view of an ultraviolet sterilization apparatus according to Embodiment 2 of the present invention;

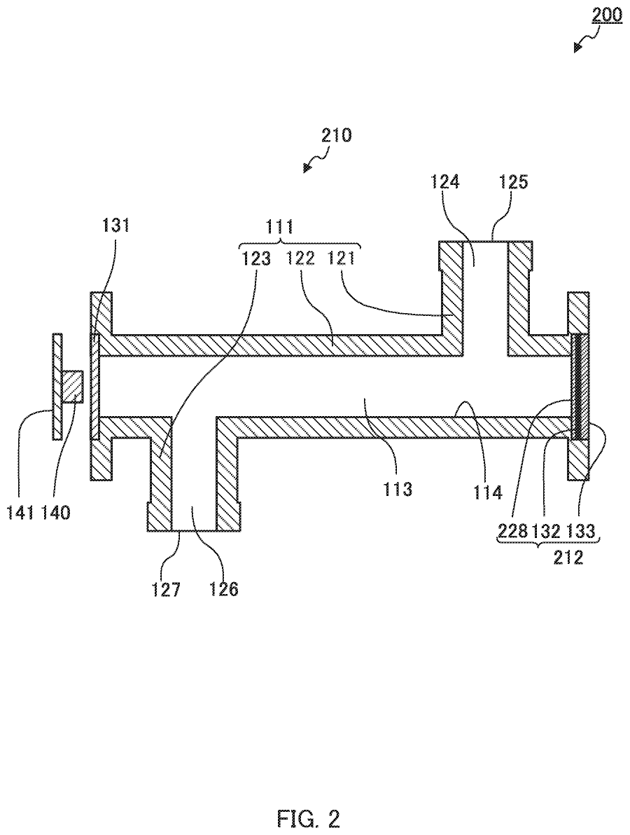

FIG. 3 is a sectional view of an ultraviolet sterilization apparatus according to Modification 1 of the present invention; and

FIG. 4 is a sectional view of an ultraviolet sterilization apparatus according to Modification 2 of the present invention.

### DESCRIPTION OF EMBODIMENTS

Embodiments of the present invention are described below in detail with reference to the accompanying drawings.

### Embodiment 1

### (Configuration of Ultraviolet Sterilization Apparatus)

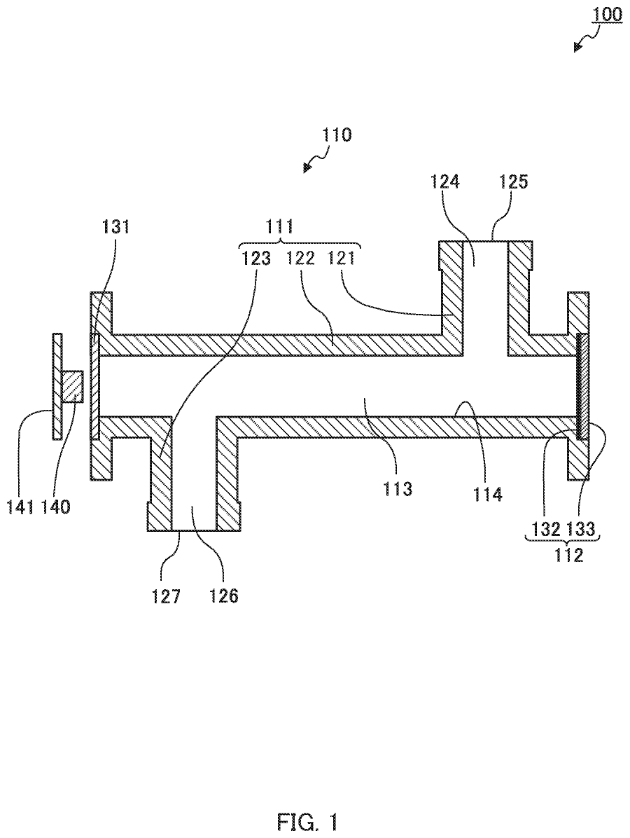

FIG. 1 is a sectional view of ultraviolet sterilizing apparatus 100 according to Embodiment 1 of the present invention.

As shown in FIG. 1, ultraviolet sterilizing apparatus 100 includes ultraviolet sterilizing pipe 110 and light source 140.

Ultraviolet sterilization pipe 110 includes channel pipe 111, window 112, and entrance window 131.

Channel pipe 111 is a pipe through which a fluid to be treated flows. Here, “fluid” means a substance which can flow through channel pipe 111, such as a liquid and a gas. Channel pipe 111 includes inflow pipe 121, channel pipe body 122, and outflow pipe 123.

Inflow pipe 121 is used for introducing a fluid to be sterilized by irradiation with ultraviolet radiation into treatment channel 113. Inflow pipe 121 has inflow channel 124 therein. The upstream end of inflow pipe 121 is inflow port 125 for allowing the fluid to flow into inflow channel 124. The downstream end of inflow pipe 121 is open to pipe wall 114 near the upstream end of channel pipe body 122. Inflow pipe 121 is connected to a fluid supply apparatus (not shown) or the like via inflow port 125 to guide the fluid from the fluid supply apparatus to treatment channel 113. Inflow port 125 may have such a shape as to allow a hose for guiding the fluid to inflow channel 124 to be fitted thereto.

Channel pipe body 122 has treatment channel 113 in which the fluid flows from one end side toward the other end side. The shape of channel pipe body 122 is not particularly limited as long as it allows the fluid to flow. The shape of channel pipe body 122 may be linear or curved. In the present embodiment, the shape of channel pipe body 122 is linear. In addition, the sectional shape of channel pipe body 122 in a direction perpendicular to the flow direction of the fluid is not particularly limited. The sectional shape may be circular or polygonal. In the present embodiment, the sectional shape of channel pipe body 122 in the direction perpendicular to the direction in which the fluid flows is circular.

Channel pipe body 122 is preferably formed of a material having a high reflectance of ultraviolet radiation. Examples of the material for channel pipe body 122 include mirror polished aluminum (Al) and polytetrafluoroethylene (PTFE). Note that the material of channel pipe body 122 is preferably PTFE as it is chemically stable and also has a higher reflectance of ultraviolet radiation. When channel pipe body 122 is formed of a material having a high reflectance of ultraviolet radiation, the efficiency of use of the ultraviolet radiation emitted from light source 140 can be increased.

The size of channel pipe body 122 is not particularly limited as long as the fluid can be sufficiently sterilized by irradiation with ultraviolet radiation. For example, when the ultraviolet radiation is emitted from light source 140 of a single lamp having a light output of 30 mW/lamp, the inner diameter of channel pipe body 122 may be 5 cm or less, and the channel length of channel pipe body 122 may be 2 cm or more and 30 cm or less. Even if the sizes of the inner diameter of the channel pipe body 122 and the length of the treatment channel 113 are different from the above, an equivalent sterilization effect can be obtained by changing the number of light source 140. In the present embodiment, window 112 is arranged on the upstream end surface of channel pipe body 122, and entrance window 131 is arranged on the downstream end surface.

Outflow pipe 123 is used to cause the sterilized fluid to flow out of treatment channel 113. Outflow pipe 123 has outflow channel 126 therein. The upstream end of outflow pipe 123 is open near the downstream end of channel pipe body 122. The downstream end of outflow pipe 123 is outflow port 127 for leading to a liquid storage apparatus (not shown) or the like. Outflow port 127 is connected to the liquid storage apparatus and guides the fluid from treatment channel 113 to the liquid storage apparatus or the like. Outflow port 127 may have such a shape as to allow a hose for guiding the fluid to the liquid storage apparatus to be fitted thereto.

Entrance window 131 guides the ultraviolet radiation emitted from light source 140 into channel pipe 111 (channel pipe body 122). The position where entrance window 131 is arranged is not particularly limited as long as it can exhibit the above-described function. In the present embodiment, entrance window 131 is arranged on the downstream end surface of channel pipe body 122. Entrance window 131 is formed of a material that transmits ultraviolet radiation and is not easily deformed or damaged by the pressure of the flowing fluid. Further, the inner surface of entrance window 131 serves as a part of the outer periphery of treatment channel 113, and prevents the fluid from flowing out from one end of channel pipe body 122. The material of entrance window 131 is preferably a material that has a high transparency to ultraviolet radiation, such as quartz (SiO₂), sapphire (Al₂O₃), or an amorphous fluorine-based resin.

Window 112 is used to determine whether ultraviolet radiation is properly emitted from light source 140. Window 112 may be arranged on at least a part of the pipe wall 114, and is preferably arranged at a position where the ultraviolet radiation emitted from light source 140 can easily reach. In the present embodiment, window 112 is arranged at the end surface of the upstream side of channel pipe body 122 to face light source 140 (entrance window 131). Window 112 includes fluorescent layer 132 and ultraviolet blocking layer 133.

Fluorescent layer 132 is arranged on the side of treatment channel 113. Fluorescent layer 132 includes a phosphor that emits visible light when irradiated with ultraviolet radiation. Examples of the phosphor include calcium tungstate, magnesium tungstate, calcium halophosphate, zinc silicate, and calcium silicate. The configuration of fluorescent layer 132 is not particularly limited as long as it can exhibit the above function. The form of fluorescent layer 132 may be a resin plate or a glass plate in which phosphor particles are dispersed, or a fluorescent coating material may be applied to the surface of ultraviolet blocking layer 133 and cured. In the present embodiment, fluorescent layer 132 is obtained by applying a fluorescent coating material on the surface of ultraviolet blocking layer 133 and curing it. The inner surface of fluorescent layer 132 serves as a part of the outer periphery of treatment channel 113, and prevents the fluid from flowing out from one end portion of the channel pipe body 122.

Ultraviolet blocking layer 133 is arranged at a position farther from treatment channel 113 than fluorescent layer 132 is. Ultraviolet blocking layer 133 blocks ultraviolet radiation and transmits visible light. The configuration of ultraviolet blocking layer 133 is not particularly limited as long as it can exhibit the above function. Ultraviolet blocking layer 133 may be formed into a plate shape using an optical material such as BK7, for example. In the present embodiment, fluorescent layer 132 is formed on a surface of the ultraviolet blocking layer 133 on the side of treatment channel 113.

Light source 140 emits ultraviolet radiation toward treatment channel 113. The type of light source 140 is not particularly limited as long as it can emit ultraviolet radiation. Examples of the light source include LEDs, mercury lamps, metal halide lamps, xenon lamps, and LDs. A center wavelength or a peak wavelength of the ultraviolet radiation emitted from light source 140 is preferably 200 nm or more and 350 nm or less. The center wavelength or the peak wavelength of the ultraviolet light emitted from light source 140 is more preferably 260 nm or more and 290 nm or less from the viewpoint of high sterilization efficiency. Light source 140 is disposed on substrate 141. Light source 140 is mounted so as to face entrance window 131.

The fluid introduced into treatment channel 113 from inflow port 125 through inflow channel 124 is irradiated with the ultraviolet radiation emitted from light source 140 through entrance window 131 while flowing through treatment channel 113, and is sterilized. Thereafter, the sterilized fluid is discharged from outflow port 127 via outflow channel 126

The fluid may be a substance that is to be sterilized and is capable of flowing through treatment channel 113, and may, for example, be water or the like in the case of a liquid. The fluid includes service water including drinking water, agricultural water, and the like, and sewage including wastewater from factories and the like.

The flow rate of the fluid may be a rate at which the fluid is sufficiently sterilized by irradiation with ultraviolet radiation while flowing through treatment channel 113, and for example, in the case of irradiating the liquid with ultraviolet radiation using light source 140 of a single lamp having a light output of 30 mW/lamp, the flow rate of the liquid is preferably 10 L/min or less. By changing the number and the arrangement of light source 140, it is possible to obtain a sterilization effect equivalent to that of the present embodiment even if the flow rate is higher than 10 L/min.

As described above, ultraviolet radiation emitted from light source 140 enters treatment channel 113 through entrance window 131. Of the ultraviolet radiation that have entered treatment channel 113, part of the ultraviolet radiation directly reaches window 112 (fluorescent layer 132). In addition, other part of the ultraviolet radiation that have entered treatment channel 113 is reflected by pipe wall 114, and then reaches window 112 (fluorescent layer 132).

Of the ultraviolet radiation that have reached window 112, part of the ultraviolet radiation reaches the phosphor. The phosphor to which the ultraviolet radiation has reached emits visible light. Of the ultraviolet radiation that has reached window 112, other part of the ultraviolet radiation may be transmitted through fluorescent layer 132 without reaching the phosphor.

Of the visible light emitted from the phosphor, part of the visible light passes through ultraviolet blocking layer 133 and is emitted to the outside. On the other hand, the ultraviolet radiation that has passed through fluorescent layer 132 is blocked by ultraviolet blocking layer 133, and is not emitted to the outside. Thus, only visible light is emitted from ultraviolet sterilizing pipe 110. Therefore, a user can confirm whether or not ultraviolet radiation is irradiated into treatment channel 113 by observing visible light from fluorescent layer 132.

(Effect)

As described above, according to ultraviolet sterilization apparatus 100 of the present embodiment, since only visible light is emitted from ultraviolet sterilization pipe 110 (ultraviolet sterilization apparatus 100), it is possible to confirm (visually) whether or not ultraviolet radiation is irradiated into treatment channel 113 simply and safely.

### Embodiment 2

Ultraviolet sterilization apparatus 200 according to Embodiment 2 differs from ultraviolet sterilization apparatus 100 according to Embodiment 1 only in the configuration of window 212. Therefore, the same components as those of ultraviolet sterilization apparatus 100 according to Embodiment 1 are denoted by the same reference signs, and description thereof is omitted.

(Configuration of Ultraviolet Sterilizing Apparatus)

Ultraviolet sterilizing apparatus 200 includes ultraviolet sterilizing pipe 210 and light source 140. Ultraviolet sterilization pipe 210 includes channel pipe 111, window 212, and entrance window 131.

In addition to fluorescent layer 132 and ultraviolet blocking layer 133, window 212 of ultraviolet sterilizing pipe 210 according to the present embodiment further includes ultraviolet transmitting layer 228. Ultraviolet transmitting layer 228 is arranged on an opposite side to ultraviolet blocking layer 133 with respect to fluorescent layer 132 to transmit ultraviolet radiation. For example, ultraviolet transmitting layer 228 may be employed in a case where the phosphor of fluorescent layer 132 dissolves in the fluid. The inner surface of ultraviolet transmitting layer 228 serves as a part of the outer periphery of treatment channel 113, and prevents the fluid from flowing out of channel pipe body 112. The material of ultraviolet transmitting layer 228 is preferably a material having a high transparency to ultraviolet radiation, such as quartz (SiO2), sapphire (Al2O), or an amorphous fluorine-based resin. Ultraviolet transmitting layer 228 and fluorescent layer 132 may be arranged with a predetermined gap therebetween or may be in close contact with each other.

(Effect)

As described above, according to ultraviolet sterilization apparatus 200 of the present embodiment, in addition to the effect of Embodiment 1, it is not necessary to consider the compatibility between the fluid and the phosphor, so that the versatility of the ultraviolet sterilization apparatus can be increased. Further, since fluorescent layer 132 does not deteriorate, durability of ultraviolet sterilization device 210 can be improved.

In order to make the illuminance distribution of the ultraviolet radiation emitted from light source 140 more uniform and to sterilize the fluid in treatment channel 113 more uniformly, treatment channel 113 may be irradiated with the ultraviolet radiation while rotating light source 140. In addition, a condensing lens or a reflector for condensing ultraviolet radiation may be provided in treatment channel 113.

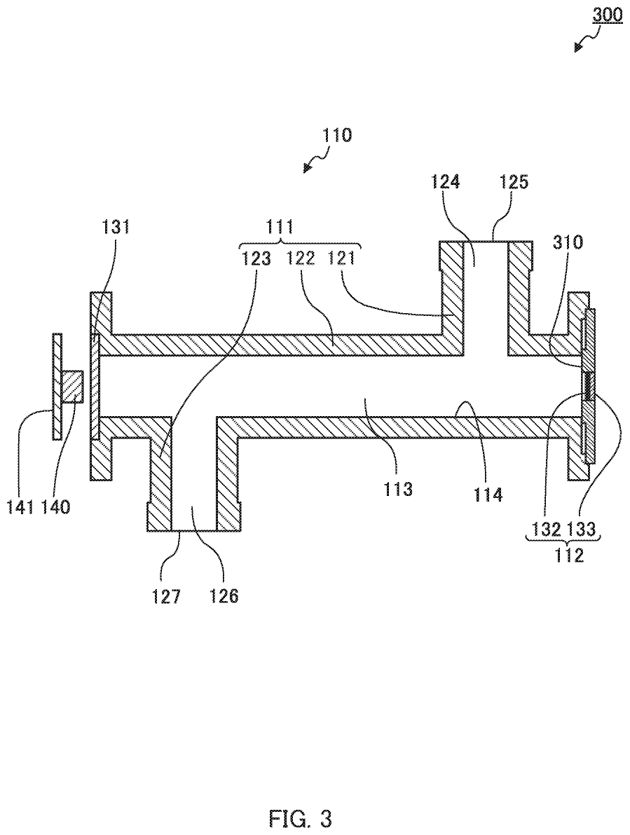

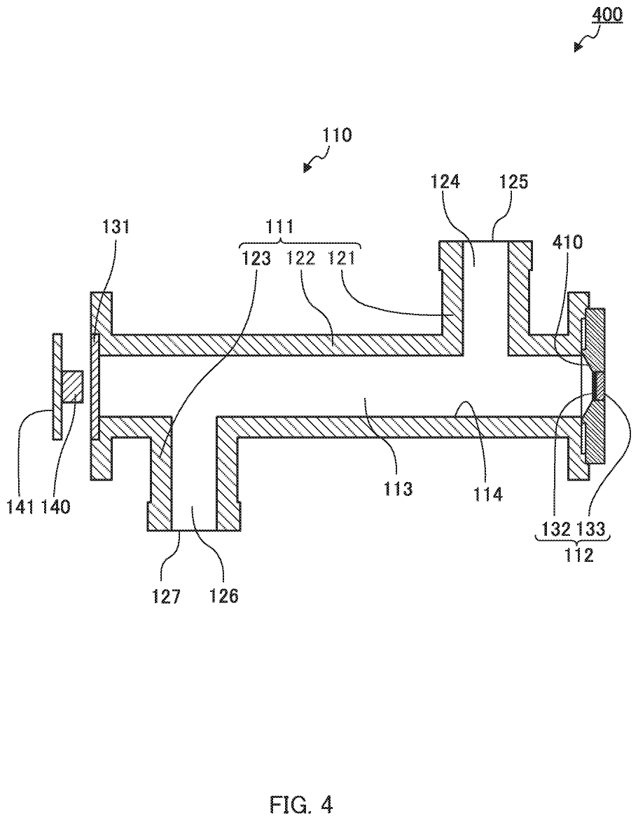

In addition, in Embodiment 1 and Embodiment 2 described above, windows 112 and 212 are arranged so as to close the entire opening on the upstream side of channel pipe body 122, but the configuration of the ultraviolet sterilization apparatus according to the present invention is not limited thereto. For example, as shown in FIGS. 3 and 4, reflection plate 310 or 410 having a through hole may be arranged so as to close the entire opening of the upstream side of flow pipe body 122, and window 112 (or window 212) may be arranged so as to close the through holes of reflection plate 310 or 410. By doing so, it is possible to utilize the ultraviolet radiation reflected by reflection plate 310 or 410 for sterilization, and it is possible to improve the sterilization efficiency. Further, since the size of windows 112 and 212 (particularly, fluorescent layer 132) can be reduced, it is also possible to reduce the manufacturing cost of the ultraviolet sterilization apparatus. The shape of the reflection surfaces of reflection plates 310 and 410 is not particularly limited, and may be a flat surface as shown in FIG. 3, or may be an inclined surface as shown in FIG. 4. The reflection plates 310 and 410 are formed of, for example, an aluminum mirror obtained by depositing aluminum on a base material, a PTFE, or the like.

The present application claims the benefit of priority based on Japanese Patent Application No. 2018-017568, filed on Feb. 2, 2018, the disclosure of which including the specification, claims, and drawings is incorporated herein by reference in its entirety.

### INDUSTRIAL APPLICABILITY

The present invention is useful, for example, for an ultraviolet sterilization apparatus for sterilization of service water, agricultural fluids, and the like.

### REFERENCE SIGNS LIST

100, 200, 300, 400 Ultraviolet sterilization apparatus
110, 210 Ultraviolet sterilization pipe
111 Channel pipe
112, 212 Window
113 Treatment channel
114 Pipe wall
121 Inflow pipe
122 Channel pipe body
123 Outflow pipe
124 Inflow channel
125 Inflow port
126 Outflow channel
127 Outflow port
131 Entrance window
132 Fluorescent layer
133 Ultraviolet blocking layer
140 Light source
141 Substrate
228 Ultraviolet transmitting layer
310, 410 Reflection plate

1. An ultraviolet sterilization pipe used for irradiating a fluid flowing through a treatment channel with ultraviolet radiation to sterilize the fluid, comprising:
a channel pipe having the treatment channel therein; and
a window arranged on at least a part of a pipe wall of the channel pipe, the window including:
a fluorescent layer including a phosphor that emits visible light when irradiated with ultraviolet radiation; and
an ultraviolet blocking layer that is arranged at a position farther from the treatment channel than the fluorescent layer is and that blocks the ultraviolet radiation and transmits the visible light.

2. The ultraviolet sterilization pipe according to claim 1,
wherein the window further includes an ultraviolet transmitting layer that is arranged on an opposite side to the ultraviolet blocking layer with respect to the fluorescent layer and that transmits the ultraviolet radiation.

3. An ultraviolet sterilization apparatus, comprising:
the ultraviolet sterilization pipe according to claim 1; and
a light source that emits the ultraviolet radiation toward the treatment channel.

4. The ultraviolet sterilization apparatus according to claim 3,
wherein the treatment channel is formed in a linear shape,
wherein the light source emits the ultraviolet radiation along an axis of the treatment channel, and
wherein the window is arranged at a position facing the light source.
